(19) United States
(12) Patent Application Publication
(10) Pub. No.: US 2008/0139655 A1
(43) Pub. Date: Jun. 12, 2008

(54) SUSTAINED-RELEASE COMPOSITION AND METHOD OF USE THEREOF

(75) Inventors: Jonathan Bortz, St. Louis, MO (US); Michael Grimshaw, St. Louis, MO (US); David F. Erkoboni, Pennington, NJ (US); Michael F. Dickus, Farmington, MO (US)

Correspondence Address: KV PHARMACEUTICAL COMPANY, 4080B WEDGEWAY COURT, EARTH CITY, MO, 63045, US

(73) Assignee: DrugTech Corporation, Wilmington, DE (US)

(21) Appl. No.: 11/850,063

(22) Filed: Sep. 5, 2007

Related U.S. Application Data
(60) Provisional application No. 60/824,985, filed on Sep. 8, 2006.
(60) Provisional application No. 60/828,276, filed on Oct. 5, 2006.

(51) Int. Cl. A61K 31/195; A61P 25/28

(52) U.S. Cl. 514/563

(57) ABSTRACT

A pharmaceutical composition comprising levodopa is provided that, when administered in a unit dosage amount of levodopa of about 100 to about 500 mg at a dosage interval of about 6 to about 24 hours, exhibits a sufficiently long release period and a sufficiently long residence time in the upper gastrointestinal tract to provide a trough concentration of levodopa in plasma of the subject that is not lower than a minimum threshold concentration below which adverse motor effects are observed in the subject. A method for treating Parkinson's disease in a subject is also provided, comprising orally administering such a composition to the subject in a unit dosage amount of levodopa of about 50 to about 1000 mg at a dosage interval of about 3 to about 24 hours.

This application claims the benefit of U.S. provisional patent application Ser. No. 60/824,985, filed on Sep. 8, 2006, and Ser. No. 60/828,276, filed on Oct. 5, 2006, the entire disclosure of each of which is incorporated by reference herein.

### FIELD OF THE INVENTION

The present invention relates to a pharmaceutical composition comprising levodopa as an active ingredient. The invention also relates to a method for treating Parkinson's disease comprising administering such a pharmaceutical composition to a subject.

### BACKGROUND OF THE INVENTION

Parkinson's disease is the second most prevalent neurodegenerative disease in the United States. There are presently an estimated 1 million Parkinson's patients with about 60,000 new cases being diagnosed annually. As the population of the United States increases, the rate of these diagnoses is expected to climb even further.

Parkinson's disease is a degenerative disorder of the central nervous system that is pathologically associated with deterioration of nuclear masses of the extrapyramidal system and a characteristic loss of melanin-containing cells from the basal ganglia, and more particularly the substantia nigra, of the brain. This loss and degeneration of dopamine producing cells results in a corresponding depletion of dopamine from the corpus striatum of the brain.

Parkinson's disease is characterized by a variety of basic symptoms including, for example, tremor, muscle rigidity, bradykinesia (and in extreme cases akinesia), and postural instability. The most apparent and well-known symptom, Parkinson's tremor, is a dyskinetic condition typically presenting as an impaired ability to control movement, e.g., a resting tremor of a patient's hand. Rigidity or stiffness and increased muscle tone, in combination with a resting tremor, can further produce a ratchety, “cogwheel” effect when a limb is passively moved. Bradykinesia is characterized as a slowness of movement and akinesia is characterized by an absence of movement. Postural instability, or a failure of postural reflexes, can lead to impaired balance and falls in a person suffering from Parkinson's.

The reduced dopamine content in the brain of patients with Parkinson's disease is considered a primary clinical cause of Parkinson's symptoms. However, dopamine is unable to cross the blood-brain barrier, preventing the use of dopamine as an effective treatment for the disease. Therefore, the treatment of Parkinson's disease is generally carried out by administering dopamine to a patient in a form of a precursor or prodrug. A classical dopamine precursor is 3,4-dihydroxy-L-phenylalanine, commonly known as levodopa or L-dopa, which corresponds to Formula (I) below.

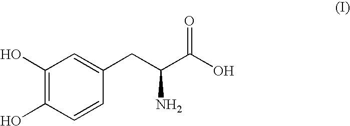

Levodopa is able to cross the blood-brain barrier and is metabolized to dopamine by decarboxylation via aromatic L-amino-acid decarboxylase. Levodopa has a short half-life of approximately 0.75 to 1.5 hours and is absorbed solely by an active amino acid transporter located in the small intestine, especially in the duodenum.

Levodopa treatment includes some well-recognized problems, however. These problems include a decline of the drug's effect after prolonged administration, a general failure to prevent progression of the disease, and significant side-effects. Levodopa-related side-effects can include levodopa-induced dyskinesias, which are extremely handicapping. Such dyskinesias can range from subtle undulating movements of the body to wild uncontrollable swinging movements of the limbs and often make it impossible for patients to perform basic tasks requiring rudimentary manual dexterity and motor coordination, e.g., feeding themselves. This and other levodopa-related motor complications can be difficult to manage and are a key source of disability for patients having Parkinson's disease.

These side effects are due in part to decarboxylation of levodopa in peripheral tissues. It is recognized that only a small portion of levodopa generally crosses the blood-brain barrier, leaving a large percentage to be metabolized elsewhere in the body of a patient. These peripheral reactions are generally mitigated through use of decarboxylase inhibitors, for example carbidopa. Carbidopa corresponds to Formula (II) below.

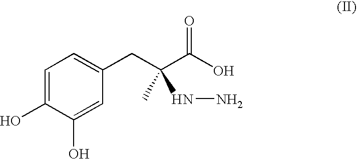

Carbidopa inhibits decarboxylation of peripheral levodopa but does not cross the blood-brain barrier or have an effect on metabolism of levodopa in the brain. As decarboxylase inhibition by carbidopa occurs only in extracerebral tissues, administration of carbidopa with levodopa increases availability of levodopa for transport to the brain.

Although a combination of carbidopa and levodopa is a commonly prescribed treatment for symptoms of Parkinson's disease, certain limitations have been recognized. As the disease progresses, typically after about three to four years of treatment, the benefit from each dose becomes shorter (the “wearing off” effect) and some patients begin to fluctuate unpredictably between mobility and immobility (the “on” and “off” effects). “On” periods are usually associated with high plasma levodopa concentrations that exceed a peak concentration in a patient and result in abnormal involuntary movements, i.e., dyskinesia. “Off” periods have been correlated with low plasma levodopa concentrations that fall below a trough concentration in a patient and result in further dyskinetic and/or bradykinetic episodes. These effects are particularly observed with intermittent or pulsatile administration of standard oral formulations of levodopa.

Pharmacokinetic profiles in human subjects of standard and controlled-release formulations of levodopa and carbidopa are known in the art. See, for example, Sagar & Smyth (2000) Analyst 125:439-445, in particular FIG. 5 thereof.

A recent study by Stocchi et al. (2005) Arch. Neurol. 62:905-910 has suggested that motor complications associated with standard levodopa therapy are related to abnormal, intermittent and/or pulsatile stimulation of denervated dopamine receptors by short-acting dopaminergic agents such as levodopa. This study involved a continuous intestinal infusion of levodopa to avoid low plasma trough levels and provide a more constant activation of brain dopamine receptors in comparison to standard oral therapy. The authors propose that motor complications related to standard methods of administering levodopa are less likely to develop when the dopaminergic therapy is delivered in a more continuous manner. They postulate that low trough levels observed with intermittent administration of standard oral formulations of levodopa cause striatal dopamine receptors to be periodically deprived of dopaminergic stimulation with consequent plastic changes in intracellular signals and neuronal firing patterns leading to motor complications. Levodopa, when administered in a manner having trough levels higher than a minimum threshold concentration, is said to have potential for improved clinical efficacy while also having higher tolerability and less severe side-effects with prolonged therapy.

Although the above study involved an intestinal infusion of levodopa, oral dosage forms exhibiting gastric retention can also provide sustained delivery of drugs. For instance, a gastric retention mechanism known as “flotation” is described in U.S. Patent Application Publication No. 2004/0180086 of Ramtoola et al. It is stated therein that flotation can be achieved with a tablet having an expandable, hydrophilic, water-permeable and substantially gas-impermeable membrane, the tablet further containing a gas-generating agent.

A dosage form having levodopa and a decarboxylase inhibitor deliverable in a hydrodynamically balanced (floating) controlled release composition is reported in U.S. Pat. No. 4,424,235 to Sheth et al.

Another means for providing sustained delivery is known as “expansion” is described, for example, in International Patent Publication No. WO 02/00213. A rapidly swelling composition, said to obstruct transit of the dosage form from the stomach to the small intestine, is reported therein. Blood concentration of levodopa is reported following administration to a dog of such a composition in a single 200 mg dose of levodopa.

Use of levodopa and carbidopa in a water-soluble or hydrophilic matrix with an organic acid is reported in U.S. Pat. No. 6,531,153 to Seth et al. The organic acid is said to chemically stabilize the carbidopa. It is further stated that the dosage form is produced by a wet granulation technique.

In U.S. Patent Application Publication No. 2004/0185097 of Kannan et al., it is reported that use of a dosage form having a release-modifying complex of polyethylene oxide provides gastric retention by expansion. It is stated therein that polyethylene oxides hydrate on exposure to water or gastric juices and swell rapidly to form hydrogels.

A gastro-retentive oral dosage form having a polymer that swells in presence of gastric fluid and gradually erodes over a period of hours is further reported in U.S. Patent Application Publication No. 2004/0185105 of Berner et al Levodopa is listed as a drug said to be deliverable by the swelling dosage form described.

Controlled release dosage forms not relying on expansion or flotation have also been described in the art. For example, in International Patent Publication No. WO 1991/16885, a controlled release dosage form is described having a wall surrounding a levodopa-containing compartment. The wall is said to comprise a polymeric composition permeable to passage of fluid and substantially impermeable to passage of levodopa. The wall is also stated to have at least one passageway for releasing levodopa under pressure from an osmotic formulation in the compartment, the osmotic formulation absorbing gastric fluid and pushing the levodopa from the dosage form.

A foldable gastro-retentive delivery system has also been described in U.S. Pat. No. 6,685,962 to Friedman et al. It is reported therein that levodopa can be administered in a dosage form having a folded configuration, for example in a capsule that unfolds after administration and is retained until dissolution in the stomach of the subject.

Klausner et al. (2003) J. Controlled Release 88:117-126 have reported a pharmacokinetic evaluation of such a gastro-retentive delivery system in dogs. Plasma concentrations of levodopa over a 12-hour period following administration of 50-200 mg dosage forms are shown graphically therein.

There remains a need for an orally deliverable pharmaceutical composition that delivers levodopa to a subject, more particularly a human subject, in need thereof in a way that reduces or minimizes motor complications. There is also a need for an improved method of treating Parkinson's disease by oral administration of levodopa.

### SUMMARY OF THE INVENTION

There is now provided an orally deliverable pharmaceutical composition comprising levodopa and at least one pharmaceutically acceptable excipient. The composition according to one embodiment satisfies the following test. When orally administered to a human subject in a unit dosage amount of levodopa of about 100 to about 500 mg at a dosage interval of about 6 to about 24 hours, the composition exhibits (a) a sufficiently long levodopa release period and (b) a sufficiently long residence time in the upper gastrointestinal tract of the subject, to provide a trough concentration of levodopa in plasma of the subject that is not lower than a minimum threshold concentration of about 300 ng/ml.

According to another embodiment, an orally deliverable pharmaceutical composition comprising levodopa and at least one pharmaceutically acceptable excipient satisfies the following test. When orally administered to a human subject in a unit dosage amount of levodopa of about 100 to about 500 mg at a dosage interval of about 8 to about 24 hours, the composition exhibits (a) a sufficiently long levodopa release period and (b) a sufficiently long residence time in the upper gastrointestinal tract of the subject, to provide a trough concentration of levodopa in plasma of the subject that is not lower than a minimum threshold concentration of about 100 ng/ml.

According to yet another embodiment, an orally deliverable pharmaceutical composition comprising levodopa and at least one pharmaceutically acceptable excipient satisfies the following test. When orally administered to a human subject in a unit dosage amount of levodopa of about 100 to about 500 mg at a dosage interval of about 6 to about 24 hours, the composition exhibits (a) a sufficiently long levodopa release period and (b) a sufficiently long residence time in the upper gastrointestinal tract of the subject, to provide a trough concentration of levodopa in plasma of the subject that is not lower than a minimum threshold concentration below which adverse motor effects are observed in the subject.

According to yet another embodiment, an orally deliverable pharmaceutical composition comprising levodopa and at least one pharmaceutically acceptable excipient satisfies the following test. When orally administered to a subject in a unit dosage amount of levodopa of about 0.5 to about 10 mg/kg body weight at a dosage interval of about 6 to about 24 hours, the composition exhibits (a) a sufficiently long levodopa release period and (b) a sufficiently long residence time in the upper gastrointestinal tract of the subject, to provide a trough concentration of levodopa in plasma of the subject that is not lower than a minimum threshold concentration of about 300 ng/ml.

A sufficiently long residence time in the upper gastrointestinal tract is achievable, for example, by a composition comprising, as pharmaceutically acceptable excipients, at least one agent that swells in presence of gastric fluid, at least one agent that generates a gas in presence of gastric fluid, and at least one agent that, in presence of gastric fluid, forms a membrane substantially impermeable to the gas. The swelling agent, gas generating agent and membrane forming agent are typically individual and separate excipients but any two or all three can optionally be pre-combined in preparation of the composition.

There is still further provided an orally deliverable pharmaceutical dosage form, comprising
(a) levodopa in an amount of about 10% to about 50% by weight;
(b) a sustained release matrix for the levodopa that (i) provides a levodopa release period substantially commensurate with a dosage interval of about 4 to about 24 hours, (ii) further functions as a swelling agent effective to cause enlargement of the dosage form in presence of gastric fluid, and (iii) comprises at least one cellulosic polymer, for example a hydroxypropoxyl-substituted cellulosic polymer;
(c) a gas generating agent in an amount effective to generate, in presence of gastric fluid, sufficient gas when entrapped in the dosage form to increase buoyancy of the dosage form in the gastric fluid; and
(d) a membrane forming agent in an amount effective to form, in presence of gastric fluid, a membrane substantially impermeable to the gas generated by the gas generating agent, such membrane trapping sufficient of the gas to increase buoyancy of the dosage form in the gastric fluid; the membrane forming agent comprising alginic acid and/or sodium alginate.
   Upon oral administration of this: dosage form in a number or fraction providing a unit dosage of levodopa of about 100 to about 500 mg to a human subject at a dosage interval in the range indicated above, the enlargement of the dosage form provided by the swelling agent and the increased buoyancy provided by the generation and entrapment of gas are together effective to enable a sufficient residence time in the upper gastrointestinal tract of the subject to provide, in combination with the levodopa release period, a trough concentration of levodopa in plasma of the subject that is not lower than a minimum threshold concentration of about 100 ng/ml or below which adverse motor effects are observed in the subject.

There is still further provided an orally deliverable pharmaceutical dosage form, comprising
(a) levodopa in an amount of about 10% to about 50% by weight;
(b) one or more cellulosic polymers, for example hydroxypropoxyl-substituted cellulosic polymers, in a total amount of about 10% to about 60% by weight;
(c) one or more mono- and/or dibasic carbonic acid salts, in a total amount of about 3% to about 15% by weight; and
(d) alginic acid and/or sodium alginate in a total amount of about 10% to about 60% by weight.

A composition or dosage form as described above optionally further comprises at least one decarboxylase inhibitor such as carbidopa, for example in an amount providing a levodopa to carbidopa ratio of about 20:1 to about 2:1 by weight.

There is still further provided a method for treating Parkinson's disease in a subject, comprising orally administering a composition or dosage form as described above to the subject in an amount providing a unit dosage of levodopa of about 50 to about 1000 mg at a dosage interval of about 3 to about 24 hours.

Such a method is adapted to provide a trough concentration of levodopa in plasma of the subject that is not lower than a minimum threshold concentration, for example a concentration of about 300 ng/ml. A minimum threshold concentration of levodopa in plasma below which adverse motor effects are observed can be defined based on individual needs of the subject, more specifically based on knowledge of a threshold concentration that is associated with adverse motor effects in the particular subject. Thus, in a still further embodiment of the invention, a method is provided for treating Parkinson's disease in a subject, comprising (a) identifying a minimum threshold of levodopa concentration in plasma of the subject below which adverse motor effects are observed, and (b) orally administering a composition or dosage form as described above to the subject in an amount providing a unit dosage of levodopa of about 50 to about 1000 mg at a dosage interval effective to provide a trough concentration of levodopa in plasma of the subject that is not lower than the minimum threshold concentration identified.

There is still further provided a method for reducing motor complications arising from Parkinson's disease therapy with levodopa, the method comprising orally administering the levodopa to the subject in a composition or dosage form as described above, in an amount providing a unit dosage of levodopa of about 50 to about 1000 mg at a dosage interval of about 3 to about 24 hours.

### DETAILED DESCRIPTION

Orally deliverable sustained-release compositions are designed to control rate of release of a drug from a dosage form to gastrointestinal fluid of a subject, where the drug typically dissolves to permit slow and/or uniform absorption of the drug over a period of time. The present compositions are believed to release the drug (levodopa) slowly during a period of residence in the upper gastrointestinal tract, for example in the stomach, delivering the drug at a relatively controlled rate to the small intestine where absorption occurs, principally in the duodenum and jejunum. According to the present invention, release rate and residence time in the upper gastrointestinal tract are controlled in such a way as to enable an absorption profile that provides a trough plasma concentration of levodopa not lower than a minimum threshold concentration as defined herein. In other words, at the end of each dosage interval, immediately prior to administration of the subsequent dose, there is still at least about 100 ng/ml levodopa in plasma of the subject, or a sufficient concentration to avoid an unacceptable degree of adverse motor effects.

With standard levodopa compositions, a short dosage interval, generally less than about 6 hours, is typically necessary to avoid the plasma concentration of levodopa falling below the threshold trough concentration. The present invention permits longer dosage intervals, for example about 6 to about 24 hours, while still maintaining trough concentrations of levodopa at or above the threshold. In various embodiments a dosage interval of about 8 to about 24 hours, about 8 to about 12 hours or about 12 to about 24 hours is permitted by compositions of the invention. Such compositions can also be administered at shorter dosage intervals if so desired, for example as short as 4 hours or even as short as 3 hours.

Previous efforts to develop a sustained-release levodopa composition have typically focused on reducing peak-to-trough variation in plasma concentration of levodopa. Compositions of the present invention are focused more specifically on eliminating the low trough levels that, according to Stocchi et al. (2005), supra, can cause striatal dopamine receptors to be periodically deprived of dopaminergic stimulation, with consequent plastic changes in intracellular signals and neuronal firing patterns, leading to motor complications.

Accordingly, a pharmaceutical composition of one embodiment of the present invention, comprising levodopa and at least one pharmaceutically acceptable excipient, when orally administered in an amount providing a unit dosage of levodopa of about 100 to about 500 mg to a human subject, for example a patient having Parkinson's disease, at a dosage interval of about 6 to about 24 hours, provides a trough concentration of levodopa in plasma of the subject that is not lower than a minimum threshold concentration of about 300 ng/ml.

A pharmaceutical composition of another embodiment of the present invention, comprising levodopa and at least one pharmaceutically acceptable excipient, when orally administered in an amount providing a unit dosage of levodopa of about 100 to about 500 mg to a human subject, for example a patient having Parkinson's disease, at a dosage interval of about 8 to about 24 hours, provides a trough concentration of levodopa in plasma of the subject that is not lower than a minimum threshold concentration of about 100 ng/ml.

A pharmaceutical composition of another embodiment of the present invention, comprising levodopa and at least one pharmaceutically acceptable excipient, when orally administered in an amount providing a unit dosage of levodopa of about 100 to about 500 mg to a human subject, for example a patient having Parkinson's disease, at a dosage interval of about 6 to about 24 hours, provides a trough concentration of levodopa in plasma of the subject that is not lower than a minimum threshold concentration below which adverse motor effects are observed in the subject.

It will be understood that the unit dosage range of about 100 to about 500 mg specified above relates specifically to a test as defined above which must be satisfied by a composition of the invention, and does not represent a limitation on unit dosages that may be useful according to methods of the invention for treating Parkinson's disease or for reducing motor complications arising from Parkinson's disease therapy with levodopa. In various embodiments the unit dosage administered according to the test is about 100 to about 400 mg, or about 150 to about 300 mg.

It will further be understood that in the test for any embodiment, a composition needs only to provide the stated trough concentration for any one unit dosage and any one dosage interval in the specified ranges to fall within the scope of that embodiment.

It will still further be understood that the minimum threshold concentration of about 300 ng/ml recited in one embodiment, or about 100 ng/ml in another embodiment, of the test as defined above does not represent a limitation on concentration below which adverse motor effects are observed in particular subjects, although it is believed that, for a majority of patients having Parkinson's disease, such adverse effects do occur when levodopa concentration is below the threshold stated. In various embodiments, the minimum threshold concentration according to the test for a composition of the invention administered at a dosage interval of about 6 to about 24 hours is about 300 ng/ml, about 350 ng/ml, about 400 ng/ml, about 450 ng/ml or about 500 ng/ml. In various embodiments, the minimum threshold concentration according to the test for a composition of the invention administered at a dosage interval of about 8 to about 24 hours is about 100 ng/ml, about 150 ng/ml, about 200 ng/ml, about 250 ng/ml, about 300 ng/ml, about 350 ng/ml, about 400 ng/ml, about 450 ng/ml or about 500 ng/ml.

It will still further be understood that compositions useful herein will provide peak levels of levodopa in plasma of the subject that preferably do not exceed a concentration that can promote adverse effects.

Unit dosages can alternatively be expressed in mg/kg body weight, where the subject is human or non-human. According to one embodiment, an orally deliverable pharmaceutical composition comprising levodopa and at least one pharmaceutically acceptable excipient satisfies the following test. When orally administered to a subject, illustratively but not necessarily a human subject, in a unit dosage amount of levodopa of about 0.5 to about 10 mg/kg body weight at a dosage interval of about 6 to about 24 hours, the composition exhibits (a) a sufficiently long levodopa release period and (b) a sufficiently long residence time in the upper gastrointestinal tract of the subject, to provide a trough concentration of levodopa in plasma of the subject that is not lower than a minimum threshold concentration of about 300 ng/ml. Illustratively, the unit dosage of levodopa used in the test for a composition of the present embodiment can be about 1 to about 10 mg/kg body weight, about 1.2 to about 8 mg/kg body weight, or about 2 to about 5 mg/kg body weight. Illustratively, the test can be conducted in a non-human species that is established as a satisfactory model for human bioavailability of levodopa, for example in a dog, especially in a beagle dog.

A trough concentration above the threshold as described for each of the embodiments described above is achieved where the composition exhibits (a) a sufficiently long levodopa release period and (b) a sufficiently long residence time in the upper gastrointestinal tract of the subject, to provide such trough concentration.

The term “trough concentration” herein means the concentration of levodopa in plasma of a subject at or near the end of a dosage interval, for example about 6 to about 24 hours after administration of the immediately preceding dose, and immediately prior to administration of the next dose, and refers to the so-called “steady-state” condition where a regimen of administration of a composition at a particular dosage interval, for example about 6 to about 24 hours, has continued for long enough to achieve substantially consistent trough levels from one dosage cycle to another. Typically such a condition is reached within about 5-10 dosage cycles. It will be understood that a trough concentration below the threshold that may be observed prior to reaching “steady-state” does not remove a method from the scope of this invention, so long as trough concentration is above the threshold after about 5-10 dosage cycles. It will further be understood that the term “steady-state” in the present context does not necessarily imply lack of variation from one dosage cycle to another; indeed trough concentration can vary substantially during a regimen of treatment but, according to the present method, is consistently above the threshold.

The minimum threshold concentration below which adverse motor effects are observed varies from subject to subject, but is typically not lower than about 100 ng/ml. In various embodiments, the minimum threshold concentration is about 100, about 150, about 200, about 250, about 300, about 350, about 400, about 450 or about 500 ng/ml.

A composition of one illustrative embodiment, when orally administered in a unit dosage amount of levodopa of about 150 to about 300 mg to a human subject at a dosage interval of about 8 to about 12 hours, exhibits (a) a sufficiently long levodopa release period and (b) a sufficiently long residence time in the upper gastrointestinal tract of the subject, to provide a trough concentration of levodopa in plasma of the subject that is not lower than a minimum threshold concentration of about 500 ng/ml.

A sufficiently long levodopa release period to achieve, in combination with a sufficiently long residence time in the upper gastrointestinal tract, a trough concentration at or above the threshold, is typically a release period that is substantially commensurate with the dosage interval. “Substantially commensurate” in the present context means at least equal to the dosage interval or not more than about 2 hours, preferably not more than about 1 hour, shorter than the dosage interval. As levodopa has a short half-life in plasma, a release period that ends more than about 2 hours prior to administration of the next dose can lead to plasma concentration falling below the threshold.

Any sustained-release mechanism known in the art can be used. Illustratively but without limitation, the levodopa can be provided in a matrix formed by a polymer that slowly erodes in gastrointestinal fluid, for example in gastric fluid, to release the levodopa over the course of the release period.

A sufficiently long residence time in the upper gastrointestinal tract to achieve, in combination with a sufficiently long levodopa release period, a steady-state trough concentration at or above the threshold, is typically a residence time that is substantially commensurate with the dosage interval, wherein “substantially commensurate” has the meaning given above. The “upper gastrointestinal tract” includes the stomach, duodenum and jejunum, but for most purposes, a composition of the invention is adapted to spend most of the residence time in the stomach.

The residence time is illustratively about 1 to about 24 hours, for example about 4 to about 24 hours. In various embodiments the composition exhibits a residence time of about 1 to about 7 hours, for example about 4 to about 7 hours; about 4 to about 12 hours; about 6 to about 24 hours; or about 8 to about 24 hours, for example about 8 hours, about 12 hours or about 24 hours.

Any mechanism known in the art for prolonging residence time in the upper gastrointestinal tract, more particularly any gastro-retentive mechanism known in the art, can be used. Illustratively but without limitation, the composition can comprise a swelling agent, for example a swellable polymer, that results in sufficient enlargement of the composition that passage through the pylorus from the stomach to the duodenum is restricted. Alternatively or in addition, the composition can have voids or can generate gas that in either case can enhance buoyancy of the composition, promoting flotation on contents of the stomach. Buoyancy can further be assisted by presence of a substantially gas-impermeable membrane that inhibits saturation of the voids or escape of the gas. Such a membrane can be present in the composition prior to administration, or can form in situ in presence of gastric fluid. The membrane should be permeable to diffusion of levodopa, to permit release of the levodopa while retaining gas for buoyancy.

Thus a suitable composition comprises a dosage form that generates gas by reaction with gastric fluid and entraps the generated gas in an amount effective to promote flotation of the dosage form in the stomach of the subject. The gas generated by reaction with gastric fluid may be, for instance, carbon dioxide liberated by reaction of a carbonate or bicarbonate salt such as calcium carbonate with acid present in the gastric fluid, for example hydrochloric acid.

The gas thus generated can be entrapped by an expandable and insoluble polymer membrane that substantially surrounds or encapsulates the dosage form. In a particular embodiment, the polymer membrane is not present as a coating or membrane prior to administration, but instead forms in the stomach of the subject following oral administration of the dosage form. The insoluble membrane allows diffusion of levodopa through the membrane to the gastrointestinal tract of the subject for absorption.

As flotation in the stomach of a subject is promoted by entrapment of gas generated in vivo, the buoyant dosage form is prevented from exiting the stomach for an extended period of time. The extended period is controllable based on the time required for the gas-generating reaction to reach completion and/or for the membrane to erode or disintegrate, thus allowing the dosage form to deflate and sink to the base of the stomach, where it can pass through the pylorus into the small intestine. Levodopa released from the dosage form during its residence in the stomach passes to the duodenum, and to a lesser extent the jejunum, of the small intestine wherein the levodopa is absorbed into the bloodstream.

In one embodiment, gastro-retention of the dosage form is promoted both by absorptive swelling of a swellable polymer matrix, which causes enlargement of the dosage form such that passage through the pylorus is restricted, and the aforementioned gas generation and entrapment, which causes flotation. Advantageously, the combination of flotation with absorptive swelling reduces risk of passage of the dosage form through the pylorus before sufficient swelling has occurred to prevent such passage. The combination also reduces risk of passage of the dosage form through the pylorus even upon gastric emptying, the occurrence of which normally renders flotation alone inadequate for gastric retention.

A composition of the invention comprises levodopa in a therapeutically effective amount for treatment of Parkinson's disease or alleviation of symptoms thereof. What constitutes a therapeutically effective amount depends on a number of factors, including the severity of the disease or symptoms, the response of the particular subject, and the desired dosage interval. For example, a composition to be administered daily, i.e., at a dosage interval of about 24 hours, generally requires a larger amount of levodopa than one to be administered more frequently, for example every 6 hours or four times a day.

A “unit dosage” of levodopa herein is an amount administered at each of the typically 1 to about 8 times (more typically 1 to about 4 times) of administration in a daily cycle. Suitable unit dosages are normally about 50 to about 1000 mg, although smaller or larger unit dosages can be found useful in particular situations. Where dosage interval is long, for example about 12 to about 24 hours, a unit dosage relatively high in the range, for example about 200 to about 1000 mg, about 200 to about 500 mg, or about 200 to about 400 mg, will generally be required; whereas where dosage interval is shorter, for example about 3 to about 8 hours, a lower unit dosage, for example about 50 to about 200 mg, or about 100 to about 200 mg, can be effective. In various embodiments a unit dosage herein is about 50, about 100, about 150, about 200, about 250, about 300, about 350, about 400 mg, about 450 mg or about 500 mg; commonly about 100 to about 400 mg and most commonly about 150 to about 300 mg. Where discrete dosage forms such as tablets or capsules are used, the unit dosage can be provided in one to a plurality of such dosage forms, or in a fraction of a dosage form. Thus, illustratively, a 200 mg unit dosage can be satisfied by administration of four 50 mg dosage forms, two 100 mg dosage forms, one 200 mg dosage form or half of one 400 mg dosage form, at each administration time in the daily cycle.

In general, levodopa is present in an amount of about 5% to about 70% by weight of the composition, more typically about 10% to about 50%, for example about 15% to about 40% or about 16% to about 30%, by weight.

At least one decarboxylase inhibitor, e.g., carbidopa, is optionally but desirably present in the composition. Decarboxylase inhibitors typically are used in a particular weight ratio with levodopa. In the case of carbidopa, a suitable ratio of levodopa to carbidopa is about 20:1 to about 2:1 by weight, although greater or smaller ratios may be useful in particular circumstances. In various embodiments the ratio is about 10:1 to about 2:1, about 6:1 to about 2.5:1 or about 4:1 to about 3:1. Carbidopa, if included, is typically present in an amount of about 1% to about 20%, for example about 2% to about 15% or about 4% to about 10%, by weight of the composition. Levodopa/carbidopa combinations for treatment of Parkinson's disease are well known and have been described, for example, in U.S. Pat. No. 4,900,755. Such a combination is commercially available as Sinemet®, and in a controlled-release formulation as Sinemet® CR, from Bristol-Myers Squibb.

Release profile of the decarboxylase inhibitor should typically match that of the levodopa to a substantial degree, so that an appropriate levodopa/carbidopa ratio is always available for absorption.

Other agents, for example monoamine oxidase (MAO) inhibitors and catechol-O-methyl transferase (COMT) inhibitors, are optionally present in the composition, in addition to or in place of a decarboxylase inhibitor. MAO inhibitors, for example selegiline or deprenyl, can block oxidative deamination of monoamines, and are known to deactivate and break down levodopa. Such agents have been shown to extend effectiveness of a levodopa dosage administered to a subject. COMT inhibitors, for example entacapone, have also been shown to prolong symptom relief by blocking conversion of levodopa by COMT to 3-O-methyldopa (3-OMD), a compound that is therapeutically ineffective and detrimental when competing with levodopa. When administered to a subject, COMT inhibitors allow larger amounts of levodopa to reach the brain of the subject. Both MAO inhibitors and COMT inhibitors contribute to higher and more consistent concentrations of levodopa in a subject's brain. If included, such inhibitors are typically present in an amount of about 1% to about 20%, for example about 2% to about 15% or about 4% to about 10%, by weight of the composition.

The invention is described herein with particular reference to an orally deliverable pharmaceutical dosage form that comprises
(a) levodopa in an amount of about 10% to about 50% by weight;
(b) a sustained release matrix for the levodopa that (i) provides a levodopa release period substantially commensurate with a dosage interval of about 4 to about 24 hours, and (ii) further functions as a swelling agent effective to cause enlargement of the dosage form in presence of gastric fluid;
(c) a gas generating agent in an amount effective to generate, in presence of gastric fluid, sufficient gas when entrapped in the dosage form to increase buoyancy of the dosage form in the gastric fluid; and
(d) a membrane forming agent in an amount effective to form, in presence of gastric fluid, a membrane substantially impermeable to the gas generated by the gas generating agent, said membrane trapping sufficient of the gas to increase buoyancy of the dosage form in the gastric fluid.
   Upon oral administration of the dosage form to a subject at a dosage interval in the range indicated above, the enlargement of the dosage form provided by the swelling agent and the increased buoyancy provided by the generation and entrapment of gas are together effective to enable a sufficient residence time in the upper gastrointestinal tract as described herein, for example a residence time that is substantially commensurate with the dosage interval.

The term “gastric fluid” as used herein means the endogenous fluid medium of the stomach, including water and secretions, for example, hydrochloric acid, or an artificial simulation of such medium, that can be useful for in vitro testing of swelling, gas generation and retention, and levodopa release properties of compositions as described herein.

The dosage form optionally further comprises a decarboxylase inhibitor as described above, for example carbidopa in an amount of about 1% to about 20% by weight.

Any pharmaceutically acceptable swellable sustained release matrix can be used. Generally, swelling agents are hydrophilic but insoluble in water, and are sometimes known as hydrogels. When hydrated, suitable swelling agents increase in volume and are elastically deformable. In their dry state, swelling agents may be structurally rigid or semi-rigid. Swelling agents include partially etherified cellulose derivatives, e.g., methylcellulose, ethylcellulose, (hydroxyethyl) methylcellulose, (hydroxypropyl) methylcellulose, (hydroxyethyl)ethylcellulose, hydroxyethylcellulose and hydroxypropylcellulose; starches; water soluble aliphatic and cyclic poly-N-vinylamides; polyvinyl alcohols; polyacrylates; polymethacrylates; polycarbophils; polyethylene glycols; and mixtures thereof.

In one embodiment the sustained release matrix comprises at least one hydroxypropoxyl-substituted cellulosic polymer, for example hydroxypropylcellulose (HPC), hydroxypropylmethylcellulose (HPMC or hypromellose) or a combination thereof, as described in greater detail below. Optionally, in this embodiment, other polymers can be present in the matrix, including for example one or more cross-linked polyacrylic acids and/or pharmaceutically acceptable salts thereof. A nonlimiting example is calcium polycarbophil, the calcium salt of polyacrylic acid cross-linked with divinyl glycol.

Any pharmaceutically acceptable gas generating agent can be used. Suitable gas generating agents are, for example, those capable of releasing carbon dioxide upon contact with gastric fluid. Such agents include, but are not limited to, pharmaceutically acceptable mono- and di-basic salts of carbonic acid, for example alkali metal and ammonium carbonates and bicarbonates, alkaline earth metal carbonates, and mixtures thereof. Representative examples of gas generating agents include sodium carbonate, sodium bicarbonate, potassium carbonate, calcium carbonate, magnesium carbonate, sodium glycine carbonate, and mixtures thereof. Such agents can, additionally or alternatively, produce carbon dioxide gas by reaction, in presence of water (e.g., upon contact with gastric fluid), with an organic acid optionally included in the composition, as described more fully below.

Any pharmaceutically acceptable membrane forming agent can be used, typically an agent that forms an insoluble polymeric membrane in the presence of gastric fluid. It should be understood that such membranes must allow diffusion of levodopa and, if included, carbidopa from the dosage form to permit absorption by the subject. Particular materials that can be useful for the present invention include polymers that are cross-linkable in the presence of gastric fluid.

Membrane forming include, for example, cellulose derivatives, dextrins, starches and starch derivatives, polymers based on carbohydrates and derivatives thereof, natural gums such as gum arabic, xanthans and alginates; polyacrylic acid, polyvinyl alcohol, polyvinyl acetate, polyvinylpyrrolidone, polymethacrylates and derivatives thereof, chitosan and derivatives thereof, and shellac and derivatives thereof.

A particularly suitable membrane forming agent comprises alginic acid and/or a salt thereof, more particularly a monovalent salt such as sodium alginate. Alginic acid is a linear copolymer with homopolymeric blocks comprised of two types of uronic acid residues, beta-D-mannuronic acid and its C₅ epimer alpha-L-guluronic acid. The uronic acids are simple monosaccharides in which the primary hydroxyl group at C₆ has been oxidized to the corresponding carboxylic acid. Polysaccharides such as alginic acid are made by linking together such monosaccharides, for example forming a long chain polymer.

A membrane is formed in vivo when alginic acid or sodium alginate is exposed to divalent cations, such as calcium (Ca²⁺) ions, in gastric fluid of a subject. Ca²⁺ ions may be donated, for example, by calcium salts such as calcium polycarbophil or calcium carbonate present in the pharmaceutical composition, and/or from calcium sources endogenous to the gastric fluid.

It is believed that polymerization of the alginate occurs because Ca²⁺ ions replace sodium ions and serve as a cross-linking agent to link two or more alginate chains together. The resulting cross-linked polymer is insoluble in gastric fluid and results in formation of an insoluble membrane substantially surrounding or encapsulating the dosage form. Such a membrane acts to entrap gas generated by the dosage form, thus promoting flotation in the stomach of the subject. Flotation in the stomach increases the residence time of the dosage form in the upper gastrointestinal tract of the subject and, in combination with a suitable release period, allows for achievement of a levodopa concentration in the plasma of the subject that remains at or above threshold at trough.

In one embodiment the dosage form comprises substantially no insoluble membrane prior to contact with gastric fluid, i.e., before administration to a subject. A dosage form having no insoluble membrane prior to contact with gastric fluid allows for rapid penetration of water to the interior of the dosage form. Such penetration allows the dosage form to quickly begin delivery of the active compound. This is advantageous over dosage forms having an insoluble membrane prior to contact with gastric fluid, as such membranes can inhibit rapid release of the active compound for absorption.

In a further aspect of the invention, an orally deliverable pharmaceutical dosage form is provided, comprising
(a) levodopa in an amount of about 10% to about 50% by weight, optionally accompanied by carbidopa in an amount of about 1% to about 20% by weight;
(b) one or more cellulosic polymers, for example hydroxypropoxyl-substituted cellulosic polymers, in a total amount of about 5% to about 60% by weight, for example about 10% to about 60% by weight;
(c) one or more mono- and/or dibasic carbonic acid salts, in a total amount of about 3% to about 15% by weight; and
(d) alginic acid and/or sodium alginate in a total amount of about 10% to about 60% by weight.

The levodopa is generally present in each dosage form in an amount of about 50 mg to about 1000 mg, more typically about 100 mg to about 800 mg or about 100 mg to about 500 mg, for example about 100, about 150, about 200, about 250, about 300, about 350, about 400, about 450 or about 500 mg, although lower or higher amounts can be useful in particular circumstances. Carbidopa, if included, is generally present in an amount of about 5 mg to about 300 mg, more typically about 30 mg to about 150 mg, for example about 30, about 45, about 60, about 75, about 90, about 105, about 120, about 135 or about 150 mg. A weight ratio of levodopa to carbidopa of about 3:1 to about 4:1 is believed to be optimal.

The one or more cellulosic polymers can comprise hydroxypropoxyl-substituted cellulosic polymers such as HPC, hypromellose or both. These polymers are available in a wide range of viscosities, and particular HPC and/or hypromellose products can be selected to meet particular needs with regard to swelling, release characteristics, and other properties. Viscosity herein is defined as 2% Brookfield viscosity (viscosity measured as a 2% solution in water at 20° C.).

Illustratively, HPC useful herein has a viscosity of about 7 to about 30,000 centipoise. Suitable HPC products are commercially available, for example under the Klucel™ trademark of Hercules, Inc., including (arranged from low to high viscosity) the Klucel E series, e.g., Klucel EF; the Klucel L series, e.g., Klucel LF; the Klucel J series, e.g., Klucel JF; the Klucel G series, e.g., Klucel GF; the Klucel M series, e.g., Klucel MF; and the Klucel H series, e.g., Klucel HF, Klucel HX and Klucel HHX. HPC can be present illustratively in an amount of about 2% to about 50%, for example about 2% to about 10% by weight of the composition.

Hypromelloses and/or HPCs of different viscosities can be used individually or in combination depending on the swelling characteristics desired for the composition. For example, hypromellose suitable for pharmaceutical compositions can be categorized as low-viscosity, medium-viscosity, and high-viscosity. Low-viscosity hypromellose useful herein has a viscosity of about 1 to about 100 centipoise, for example about 2 to about 10, illustratively about 3 centipoise. Medium-viscosity hypromellose useful herein has a viscosity of about 1,000 to about 20,000 centipoise, for example about 2,000 to about 10,000, illustratively about 4,000 centipoise. High-viscosity hypromellose useful herein has a viscosity of about 50,000 to about 500,000 centipoise, for example about 70,000 to about 200,000, illustratively about 100,000 centipoise.

Likewise, HPC suitable for pharmaceutical compositions is available in a wide range of viscosities. Illustratively, HPCs useful herein can have viscosity ranges of about 7 to about 12 centipoise (e.g., Klucel EF, Klucel LF), about 15 to about 25 centipoise (e.g., Klucel JF), about 50 to about 500 centipoise (e.g., Klucel GF), about 1,000 to about 8,000 (e.g., Klucel MF), or about 10,000 to about 30,000 (e.g., Klucel HF).

Hypromelloses further vary in degree of substitution of available hydroxyl groups on the cellulosic backbone with methoxyl groups and hydroxypropoxyl groups. With increasing hydroxypropoxyl substitution, the resulting hypromellose becomes more hydrophilic in nature. As nonlimiting examples, hypromelloses useful herein can have about 15% to about 35% methoxyl substitution, and up to about 15% hydroxypropoxyl substitution.

Suitable hypromellose products are commercially available, for example under the Methocel™ trademark of Dow Chemical Co., including Methocel E3, Methocel E4M and Methocel K100M. Hypromellose can be present illustratively in an amount of about 8% to about 50% by weight of the composition. In one embodiment, a plurality of hypromelloses of differing viscosities are present in the dosage form. For example, a dosage form can comprise about 2% to about 50% by weight, independently, of each of a low-viscosity, medium-viscosity and high-viscosity hypromellose; illustratively, about 2% to about 35% by weight of a low-viscosity hypromellose, about 2% to about 25% by weight of a medium-viscosity hypromellose, and about 2% to about 15% by weight of a high-viscosity hypromellose.

In one embodiment, the composition comprises at least two hydroxypropoxyl-substituted cellulosic polymers, at least one of which is of low viscosity (e.g., a low-viscosity hypromellose such as Methocel™ E3 and/or a low-viscosity HPC such as Klucel™ EF) and at least one of which is of high viscosity (e.g., a high-viscosity hypromellose such as Methocel™ K100M and/or a high-viscosity HPC such as Klucel™ HF or Klucel™ HX). In this embodiment, a medium-viscosity hydroxypropoxyl-substituted cellulosic polymer (e.g., a medium-viscosity hypromellose such as Methocel™ E4M and/or a medium-viscosity HPC such as Klucel™ MF) can optionally be present. Low-viscosity and high-viscosity polymers are typically present in compositions of this embodiment in a total amount of about 5% to about 25% by weight, and in a weight ratio of low-viscosity to high-viscosity polymers of about 1:5 to about 5:1, for example about 1:2 to about 2:1.

Optionally an additional swellable polymer can be present, for example calcium polycarbophil as indicated above. In one embodiment, calcium polycarbophil is present in an amount of about 5% to about 30% by weight of the composition.

In a particular embodiment, the one or more mono- and/or dibasic carbonic acid salts comprise calcium carbonate, in an amount from about 3% to about 15% by weight of the composition.

In addition to the components mentioned above, the present dosage form optionally comprises one or more additional excipients such as organic acids, wetting agents, flow regulating agents (also referred to herein as flow aids), lubricants and diluents.

Pharmaceutically acceptable organic acids, for example, citric acid, fumaric acid, malic acid, glutamic acid, succinic acid or tartaric acid, can increase evolution of gas from a gas generating agent such as calcium carbonate. However, it is believed that, according to certain embodiments of the invention, an acid component such as the above or a salt thereof, or an inorganic acid such as sodium dihydrogen phosphate or disodium hydrogen phosphate, is not necessary for effective gas generation and that endogenous acids in the stomach of a subject are sufficient.

In particular embodiments, however, organic acids can have advantages in chemically stabilizing carbidopa, where this is included as a decarboxylase inhibitor. Illustratively, at least one organic acid can be present in a total organic acid amount of about 1.5% to about 6% by weight of the composition.

The present dosage form optionally comprises one or more pharmaceutically acceptable wetting agents or surfactants. These agents are believed to promote close association of levodopa with water, thereby enhancing dissolution in gastrointestinal fluid, a condition that is believed to improve bioavailability of the drug.

Suitable surfactants include anionic and nonionic surfactants. Anionic surfactants include alkyl sulfates, e.g., sodium, potassium or magnesium n-dodecyl (lauryl) sulfate, n-tetradecyl sulfate, n-hexadecyl sulfate or n-octadecyl sulfate; alkyl ether sulfates, e.g., sodium, potassium or magnesium n-dodecyloxyethyl sulfate or n-tetradecyloxyethyl sulfate; or alkanesulfonates, e.g., sodium, potassium or magnesium n-dodecanesulfonate, n-tetradecanesulfonate, n-hexadecanesulfonate or n-octadecane-sulfonate. Nonionic surfactants include fatty acid/polyhydroxyl alcohol esters, e.g., sorbitan monolaurate, monooleate, monostearate, monopalmitate, tristearate or trioleate; polyoxyethylene adducts of fatty acid/polyhydroxyl alcohol esters, e.g., polyoxyethylene sorbitan monolaurate, monooleate, monostearate, monopalinitate, tristearate or trioleate; polyethylene glycol/fatty acid esters, e.g., polyoxyethylene stearate, polyethylene glycol 400 stearate or polyethylene glycol 2000 stearate; ethylene oxide/propylene oxide block copolymers; myristates and their condensation products; or ethylene oxide homopolymers having a degree of polymerization of about 2,000 to about 100,000, as commercially available for example under the trademark Polyox® of Union Carbide.

Particularly suitable wetting agents include, either individually or in combination, oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, and sodium lauryl sulfate.

Wetting agents if included are illustratively present in an amount of about 0.05% to about 10%, for example about 0.1% to about 5%, or about 0.5% to about 2%, by weight of the composition. In one particular embodiment, sodium lauryl sulfate is present in an amount of about 0.5% to about 1.5% by weight of the composition.

The present dosage form optionally comprises one or more pharmaceutically acceptable flow aids, lubricants and/or glidants, which can be useful in facilitating preparation of the dosage form. In one embodiment, at least one flow aid is present in the dosage form in a total flow aid amount of about 0.05% to about 5%, for example about 0.1% to about 4%, by weight of the composition. Suitable flow aids include without limitation talc, modified and unmodified starches, colloidal silica and mixtures thereof. In one embodiment, at least one lubricant and/or glidant is present in the dosage form in a total lubricant and glidant amount of about 0.5% to about 5% by weight of the composition. Suitable lubricants and/or glidants include without limitation glyceryl tribehenate; stearates, e.g. magnesium, calcium and sodium stearates; stearic acid; sodium stearyl fumarate; hydrogenated vegetable oils; talc; waxes; boric acid; sodium benzoate; sodium acetate; sodium chloride; DL-leucine; polyethylene glycols, e.g., Carbowax™ 4000 and Carbowax™ 6000; and mixtures thereof. In one embodiment at least one lubricant is present in the dosage form, selected from magnesium stearate, calcium stearate, stearic acid, sodium stearyl fumarate and combinations thereof.

A diluent is a substance added to increase the bulk of a mixture to make a dosage form such as a tablet of a practical size when only a small amount of active ingredient and functional excipients is present. Suitable diluents include, as nonlimiting examples, lactose, cellulose, dry starch, powdered sugar, dicalcium phosphate, calcium sulfate, sodium chloride, kaolin, mannitol, sorbitol, sucrose and inositol. In general, the present dosage forms contain a sufficiently large amount of functional excipients such as a swelling agent, gas generating agent and membrane forming agent that a diluent is often unnecessary.

A pharmaceutical composition or dosage form as provided herein can optionally include one or more further active pharmaceutical and/or nutritional compounds addressing medical conditions and symptoms associated with Parkinson's disease, such as depression, autonomic neuropathy and/or sleep disorders. Such compounds are well known and may be selected by one of ordinary skill depending upon the needs of an individual subject.

A dosage form of the present invention can take the form, for example, of a tablet, hard or soft capsule or pill, or granules, for example coated granules. Any dosage form generally suitable for oral administration of a swallowable sustained-release composition can be used. Such dosage forms can be prepared by methods known in the art for preparing oral dosage forms, including direct compression, wet granulation, dry granulation and encapsulation. In one particular embodiment, the dosage form comprises a tablet formed by direct compression. Conventional direct compression techniques known to those of ordinary skill in the art can be employed. Tablets useful herein can be coated or uncoated. A tablet coating if provided is, in one embodiment, of sufficient thickness and strength to provide acceptable tablet integrity during storage, transportation and administration, but not to substantially inhibit or delay uptake of water from gastric fluid as may be required for swelling and evolution of gas.

A dosage form such as a tablet is illustratively of such a size and shape that, upon swelling in the gastric fluid, it does not readily or immediately pass through the pylorus into the duodenum. Suitably, for example, a round tablet having flat or convex opposing faces can have the following dimensions when dry: diameter about 1 to about 1.5 cm; maximum depth about 0.5 to about 1 cm.

A dosage form of the invention is exemplified by a tablet comprising, by weight,
about 16% to about 30% levodopa;
about 4% to about 10% carbidopa;
about 2% to about 35% low-viscosity hypromellose (about 1 to about 5 centipoise);
about 2% to about 25% medium-viscosity hypromellose (about 2,000 to about 6,000 centipoise);
about 2% to about 15% high-viscosity hypromellose (about 75,000 to about 125,000 centipoise);
about 2% to about 10% HPC;
about 5% to about 30% calcium polycarbophil;
about 3% to about 15% calcium carbonate; and
about 15% to about 50% sodium alginate.

Such a tablet optionally further comprises, by weight, one or more of
about 0.75% to about 3% fumaric acid;
about 0.75% to about 3% citric acid;
about 0.1% to about 0.3% sodium lauryl sulfate;
about 0.1% to about 0.3% silicon dioxide;
about 0.5% to about 1.5% sodium stearyl fumarate;
about 0.5% to about 1% magnesium stearate; and
about 0.5% to about 2% stearic acid.

A dosage form of the invention is further exemplified by a tablet comprising, by weight,
about 16% to about 30% levodopa;
about 4% to about 10% carbidopa;
about 5% to about 20% total hydroxypropoxyl-substituted cellulosic polymers, including polymers selected from each of (a) low-viscosity hypromelloses (about 1 to about 5 centipoise) or HPCs (about 7 to about 12 centipoise), and (b) high-viscosity hypromelloses (about 75,000 to about 125,000 centipoise) or HPCs (about 10,000 to about 30,000 centipoise);
about 3% to about 15% calcium carbonate;
about 15% to about 50% sodium alginate;
about 0.5% to about 5% total organic acid, comprising one or more of citric, fumaric, malic, glutamic, succinic and tartaric acids;
about 0.1% to about 0.3% sodium lauryl sulfate;
about 0.1% to about 0.3% silicon dioxide;
about 0.5% to about 1% magnesium stearate; and
about 0.5% to about 2% stearic acid.

The present invention further provides a method for treating Parkinson's disease in a subject in need of such treatment. The method comprises orally administering a composition or dosage form as described herein to the subject in an amount providing a unit dosage of levodopa of about 50 to about 1000 mg at a dosage interval of about 3 to about 24 hours. In a closely related embodiment, the invention further provides use of a composition as described herein in preparation of a medicament for treating Parkinson's disease in a subject by administering to the subject an amount of the composition providing a unit dosage of levodopa of about 50 to about 1000 mg at a dosage interval of about 3 to about 24 hours.

Unless the context demands otherwise, the term “treat,” “treating” or “treatment” herein includes preventive or prophylactic use of an agent, in the present instance levodopa, in a subject having Parkinson's disease and at risk of developing symptoms associated therewith, as well as use of such an agent in a subject already experiencing Parkinson's symptoms, as a therapy to alleviate, relieve, reduce intensity of or eliminate such symptoms or an underlying cause thereof.

The term “subject” refers to a warm-blooded animal, generally a mammal such as, for example, a cat, dog, horse, cow, pig, mouse, rat or primate, including a human. In one particular embodiment the subject is a human, for example, a patient having Parkinson's disease.

In illustrative embodiments, the dosage interval is about 4 to about 24 hours, about 4 to about 7 hours, about 6 to about 24 hours, about 8 to about 24 hours, or about 12 to about 24 hours, for example about 8, about 12 or about 24 hours. Where the dosage interval is about 8 hours, administration is three times daily; where the dosage interval is about 12 hours, administration is twice daily; and where the dosage interval is about 24 hours, administration is once daily.

Where necessary to deliver a sufficient dose of levodopa, more than one dosage form can be administered at each dosage time. Total levodopa dosage at each dosage time is generally about 50 mg to about 3000 mg. More typically, the levodopa dosage is about 100 mg to about 1000 mg, for example about 100 mg to about 500 mg or about 100 mg to about 400 mg, and can be delivered in a single sustained-release dosage form, for example comprising about 100, about 150, about 200, about 250, about 300, about 350, about 400, about 450 or about 500 mg levodopa. As indicated above, a decarboxylase inhibitor such as carbidopa is optionally present in the dosage form.

Dosage amount of levodopa should not be so high as to provide peak levels of levodopa or any metabolite thereof in plasma of the subject that can result in adverse effects.

The present invention further provides a method for treating Parkinson's disease in a subject, comprising
(a) identifying a minimum threshold of levodopa concentration in plasma of the subject below which adverse motor effects are observed, and
(b) orally administering a composition or dosage form as described herein to the subject in an amount providing a unit dosage of levodopa of about 50 to about 1000 mg at a dosage interval effective to provide a trough concentration of levodopa in plasma of the subject that is not lower than the minimum threshold concentration identified.

Once a minimum threshold concentration of levodopa is identified, a composition of the invention is orally administered (step (b) above) in accordance with the method previously described.

Identifying the threshold for a particular subject in step (a) above can be done, for example, by
(i) measuring concentration of levodopa in plasma of the subject during at least one period of levodopa administration;
(ii) recording frequency and/or intensity of adverse motor effects in the subject during the at least one period of levodopa administration;
(iii) correlating the concentration of levodopa in plasma with the frequency and/or intensity of adverse motor effects in the subject; and
(iv) identifying from the resulting correlation a minimum threshold concentration below which adverse motor effects occur at an unacceptable frequency or intensity.

The composition or dosage form should be selected to have a levodopa release period and a residence time in the upper gastrointestinal tract substantially commensurate with the dosage interval. Selection of the composition or dosage form should also take into account the minimum threshold concentration of levodopa for the subject, below which adverse motor effects are likely to occur. One of skill in the art may recognize additional or different reasons for selecting a minimum threshold concentration to be provided by the composition or dosage form. The threshold is typically not lower than about 100 ng/ml. In various embodiments, the minimum threshold concentration is about 100, about 150, about 200, about 250, about 300, about 350, about 400, about 450 or about 500 ng/ml.

A treatment regimen comprising oral administration of a composition or dosage form of the invention in a suitable unit dosage at a suitable dosage interval can be continued for as long as determined by the physician, for example for a period of about one month to about five years or for substantially the remainder of the life of the subject. Selection of a particular dosage form, unit dosage and dosage interval can be repeated at any time during the regimen.

Methods of the present invention enable avoidance of the low trough levels of levodopa that are observed with intermittent or pulsatile administration of standard oral formulations of levodopa. Periodic deprivation of dopaminergic stimulation associated with those low trough levels are thereby reduced or eliminated, leading to reduction or elimination of adverse motor complications in subjects being treated for Parkinson's disease. Accordingly, oral administration of a levodopa composition of the present invention can provide improved clinical efficacy, higher tolerability and less severe side-effects.

### EXAMPLES

The following examples are merely illustrative and do not limit this disclosure in any way.

### Example 1

Tablet formulations A and B are prepared comprising ingredients in ranges of amounts as shown in Table 1.

TABLE 1
Compositions of tablet formulations A and B (weights in mg)

| Ingredient | A | B |
|---|---|---|
| 1. levodopa | 150-300 | 150-300 |
| 2. carbidopa | 40-80 | 40-80 |
| 3. silicon dioxide | 1-2.5 | 1-2.5 |
| 4. high-viscosity hypromellose, e.g., | 25-100 | 0 |
| Methocel ™ K100M | | |
| 5. low-viscosity hypromellose, e.g., | 20-50 | 20-50 |
| Methocel ™ E3LV | | |
| 6. high-viscosity HPC, e.g., Klucel ™ HHX | | 25-100 |
| 7. sodium alginate LFR 5/60 | 200-400 | 200-400 |
| 8. calcium carbonate USP, heavy | 25-100 | 25-100 |
| 9. sodium lauryl sulfate | 1-2.5 | 1-2.5 |
| 10. organic acid* | 5-20 | 5-20 |
| 11. stearic acid, purified | 5-10 | 5-10 |
| 12. magnesium stearate | 2.5-5 | 2.5-5 |
| Total weight | 500-1000 | 500-1000 |

*total of one or more of citric, fumaric, malic, glutamic, succinic and tartaric acids

If included, high-viscosity hypromellose (item 4) is prepared as a 10% solution in water. Items 1-8 are charged to a suitable twin-shell blender and blended for about 10 minutes. The resulting blend is charged to a suitable fluid bed granulator with a top spray and granulated with minimal fluidization. The resulting granulated mixture is dried to a moisture content not less than 10%, and is then co-milled, for example through a 39 mesh screen at 1,400 rpm. The co-milled granulated mixture is then dispensed in a proper amount and charged to a suitable twin-shell blender. Items 9 and 10 are added, for example through a 20 mesh screen, and blended for about 10 minutes. Items 11 and 12 are then added, for example through a 20 mesh screen, and blended for about 3 minutes. The resulting blend is compressed on a rotary tablet press, for example to a hardness of 7 to 9 Strong-Cobb units.

### Example 2

Tablet formulations A and B prepared as above are subjected to dissolution testing for both levodopa and carbidopa (USP apparatus II with paddles, 50 rpm, dissolution medium 900 ml 0.1N HCl) over a 12 hour period. Results for both formulations are typically in the ranges shown in Table 2.

TABLE 2
Dissolution of tablet formulations

| Ingredient | Time (h) | % Dissolved |
|---|---|---|
| levodopa | 1 | 15-25 |
| | 2 | 25-40 |
| | 4 | 40-70 |
| | 8 | 65-80 |
| | 12 | 80-95 |
| carbidopa | 1 | 15-25 |
| | 2 | 25-40 |
| | 4 | 40-70 |
| | 8 | 65-80 |
| | 12 | 80-95 |

The words “comprise”, “comprises”, and “comprising are to be interpreted inclusively rather than exclusively.

All patents and publication cited herein are incorporated by reference into this application in their entirety.

What is claimed is:

1. An orally deliverable pharmaceutical composition comprising levodopa and at least one pharmaceutically acceptable excipient; the composition, when orally administered in a unit dosage amount of levodopa of about 100 to about 500 mg to a human subject at a dosage interval of about 6 to about 24 hours, exhibiting (a) a sufficiently long levodopa release period and (b) a sufficiently long residence time in the upper gastrointestinal tract of the subject, to provide a trough concentration of levodopa in plasma of the subject that is not lower than a minimum threshold concentration of about 300 ng/ml.

2. The composition of claim 1, wherein the release period and residence time are sufficiently long to provide a trough concentration that is not lower than a minimum threshold concentration of about 500 ng/ml.

3. The composition of claim 1, wherein the unit dosage amount of levodopa is about 150 to about 300 mg.

4. The composition of claim 1, wherein the dosage interval is about 8 to about 12 hours.

5. The composition of claim 1 that, when orally administered in a unit dosage amount of levodopa of about 150 to about 300 mg to a human subject at a dosage interval of about 8 to about 12 hours, exhibits (a) a sufficiently long levodopa release period and (b) a sufficiently long residence time in the upper gastrointestinal tract of the subject, to provide a trough concentration of levodopa in plasma of the subject that is not lower than a minimum threshold concentration of about 500 ng/ml.

6. The composition of claim 1, wherein the at least one excipient comprises (a) at least one agent that swells in presence of gastric fluid, (b) at least one agent that generates a gas in presence of gastric fluid, and (c) at least one agent that, in presence of gastric fluid, forms a membrane substantially impermeable to the gas.

7. The composition of claim 6, wherein the at least one swelling agent swells from absorption of gastric fluid.

8. The composition of claim 6, wherein the at least one gas generating agent reacts with gastric fluid to produce a gas.

9. The composition of claim 8, wherein the gas generated is carbon dioxide.

10. The composition of claim 6, further comprising an organic acid; wherein the at least one gas generating agent reacts with the organic acid in presence of water to produce carbon dioxide gas.

11. The composition of claim 6, wherein the membrane formed in presence of gastric fluid by the at least one membrane forming agent is a substantially insoluble polymer membrane.

12. The composition of claim 11, wherein the membrane is permeable to diffusion of the levodopa.

13. The composition of claim 1, further comprising at least one decarboxylase inhibitor.

14. The composition of claim 13, wherein the at least one decarboxylase inhibitor comprises carbidopa.

15. The composition of claim 14, having a levodopa to carbidopa ratio of about 20:1 to about 2:1 by weight.

16. An orally deliverable pharmaceutical composition comprising levodopa and at least one pharmaceutically acceptable excipient; the composition, when orally administered in a unit dosage amount of levodopa of about 100 to about 500 mg to a human subject at a dosage interval of about 8 to about 24 hours, exhibiting (a) a sufficiently long levodopa release period and (b) a sufficiently long residence time in the upper gastrointestinal tract of the subject, to provide a trough concentration of levodopa in plasma of the subject that is not lower than a minimum threshold concentration of about 100 ng/ml.

17. An orally deliverable pharmaceutical composition comprising levodopa and at least one pharmaceutically acceptable excipient; the composition, when orally administered in a unit dosage amount of levodopa of about 100 to about 500 mg to a human subject at a dosage interval of about 6 to about 24 hours, exhibiting (a) a sufficiently long levodopa release period and (b) a sufficiently long residence time in the upper gastrointestinal tract of the subject, to provide a trough concentration of levodopa in plasma of the subject that is not lower than a minimum threshold concentration below which adverse motor effects are observed in the subject.

18. An orally deliverable pharmaceutical composition comprising levodopa and at least one pharmaceutically acceptable excipient; the composition, when orally administered to a subject in a unit dosage amount of levodopa of about 0.5 to about 10 mg/kg body weight at a dosage interval of about 6 to about 24 hours, exhibiting (a) a sufficiently long levodopa release period and (b) a sufficiently long residence time in the upper gastrointestinal tract of the subject, to provide a trough concentration of levodopa in plasma of the subject that is not lower than a minimum threshold concentration of about 300 ng/ml.

19. The composition of claim 18, wherein the unit dosage amount of levodopa is about 1.2 to about 8 mg/kg.

20. The composition of claim 18, wherein the subject is a non-human animal model for human bioavailability of levodopa.

21. The composition of claim 18, wherein the subject is a dog.

22. An orally deliverable pharmaceutical dosage form comprising
(a) levodopa in an amount of about 10% to about 50% by weight;
(b) a sustained release matrix for the levodopa that (i) provides a levodopa release period substantially commensurate with a dosage interval of about 4 to about 24 hours, (ii) further functions as a swelling agent effective to cause enlargement of the dosage form in presence of gastric fluid, and (iii) comprises at least one cellulosic polymer;
(c) a gas generating agent in an amount effective to generate, in presence of gastric fluid, sufficient gas when entrapped in the dosage form to increase buoyancy of the dosage form in the gastric fluid; and
(d) a membrane forming agent in an amount effective to form, in presence of gastric fluid, a membrane substantially impermeable to the gas generated by the gas generating agent, said membrane trapping sufficient of the gas to increase buoyancy of the dosage form in the gastric fluid; the membrane forming agent comprising alginic acid and/or sodium alginate;
wherein, upon oral administration of the dosage form in a number or fraction providing a unit dosage of levodopa of about 100 to about 500 mg to a human subject at said dosage interval, the enlargement of the dosage form provided by the swelling agent and the increased buoyancy provided by the generation and entrapment of gas are together effective to enable a sufficient residence time in the upper gastrointestinal tract of the subject to provide, in combination with the levodopa release period, a trough concentration of levodopa in plasma of the subject that is not lower than a minimum threshold concentration of about 100 ng/ml or below which adverse motor effects are observed in the subject.

23. The dosage form of claim 22, wherein the release period and residence time are sufficiently long to provide a trough concentration that is not lower than a minimum threshold concentration of about 300 ng/ml.

24. The dosage form of claim 22, wherein the release period and residence time are sufficiently long to provide a trough concentration that is not lower than a minimum threshold concentration of about 500 ng/ml.

25. The dosage form of claim 22, comprising about 50 to about 500 mg levodopa.

26. The dosage form of claim 22, comprising about 100 to about 400 mg levodopa.

27. The dosage form of claim 22, wherein the at least one cellulosic polymer comprises at least one hydroxypropoxyl-substituted cellulosic polymer.

28. The dosage form of claim 22, comprising substantially no gas-impermeable membrane prior to exposure of the dosage form to gastric fluid.

29. The dosage form of claim 22, further comprising at least one decarboxylase inhibitor.

30. The dosage form of claim 29, wherein the at least one decarboxylase inhibitor comprises carbidopa.

31. The dosage form of claim 30, having a levodopa to carbidopa ratio of about 20:1 to about 2:1 by weight.

32. The dosage form of claim 22, exhibiting a residence time in the upper gastrointestinal tract of about 1 to about 24 hours.

33. The dosage form of claim 32, wherein said residence time is about 4 to about 24 hours.

34. The dosage form of claim 32, wherein said residence time is about 1 to about 7 hours.

35. The dosage form of claim 32, wherein said residence time is about 8 to about 24 hours.

36. An orally deliverable pharmaceutical dosage form, comprising
(a) levodopa in an amount of about 10% to about 50% by weight;
(b) one or more cellulosic polymers, in a total amount of about 5% to about 60% by weight;
(c) one or more mono- and/or dibasic carbonic acid salts, in a total amount of about 3% to about 15% by weight; and
(d) alginic acid and/or sodium alginate in a total amount of about 10% to about 60% by weight.

37. The dosage form of claim 36, comprising about 50 to about 500 mg levodopa.

38. The dosage form of claim 36, comprising about 100 to about 400 mg levodopa.

39. The dosage form of claim 36, wherein the levodopa is present in an amount of about 16% to about 30% by weight.

40. The dosage form of claim 36, further comprising carbidopa in an amount of about 1% to about 20% by weight.

41. The dosage form of claim 40, wherein the carbidopa is present in an amount of about 4% to about 10% by weight.

42. The dosage form of claim 40, further comprising at least one organic acid in an amount effective to stabilize the carbidopa.

43. The dosage form of claim 42, wherein the at least one organic acid is present in a total amount of about 1.5% to about 6% by weight.

44. The dosage form of claim 42, wherein the at least one organic acid comprises citric acid, fumaric acid, malic acid, glutamic acid, succinic acid, tartaric acid or a combination thereof.

45. The dosage form of claim 36, further comprising at least one wetting agent in a total amount of about 0.05% to about 1% by weight.

46. The dosage form of claim 45, wherein the at least one wetting agent is present in a total amount of about 0.1% to about 0.5% by weight.

47. The dosage form of claim 45, wherein the wetting agent comprises sodium lauryl sulfate.

48. The dosage form of claim 36, further comprising at least one flow aid and/or at least one lubricant.

49. The dosage form of claim 48, comprising at least one flow aid in a total amount of about 0.05% to about 5% by weight.

50. The dosage form of claim 49, wherein the at least one flow aid comprises silicon dioxide.

51. The dosage form of claim 48, comprising at least one lubricant in a total amount of about 0.5% to about 5% by weight.

52. The dosage form of claim 51, wherein the at least one lubricant comprises magnesium stearate, calcium stearate, stearic acid, sodium stearyl fumarate or a combination thereof.

53. The dosage form of claim 36, wherein the one or more cellulosic polymers comprise one or more hydroxypropoxyl-substituted cellulosic polymers.

54. The dosage form of claim 53, wherein the one or more hydroxypropoxyl-substituted cellulosic polymers comprise hypromellose, HPC or a combination thereof.

55. The dosage form of claim 53, comprising a plurality of hydroxypropoxyl-substituted cellulosic polymers having differing viscosities.

56. The dosage form of claim 55, comprising at least two hydroxypropoxyl-substituted cellulosic polymers, at least one of which is of low viscosity and at least one of which is of high viscosity, in a total hydroxypropoxyl-substituted cellulosic polymer amount of about 5% to about 25% by weight, and in a weight ratio of low-viscosity to high-viscosity polymers of about 1:5 to about 5:1.

57. The dosage form of claim 36, further comprising calcium polycarbophil in an amount of about 5% to about 30% by weight.

58. The dosage form of claim 36, wherein the one or more mono- and/or dibasic carbonic acid salts comprise calcium carbonate.

59. The dosage form of claim 36, wherein the alginic acid and/or sodium alginate are present in a total amount of about 15% to about 50% by weight.

60. The dosage form of claim 36, that is a tablet.

61. The dosage form of claim 60, having no substantially insoluble membrane prior to being administered to a subject.

62. The dosage form of claim 36, comprising
about 16% to about 30% levodopa;
about 4% to about 10% carbidopa;
about 5% to about 25% total hydroxypropoxyl-substituted cellulosic polymers, including polymers selected from (a) low-viscosity hypromelloses and HPCs and (b) high-viscosity hypromelloses and HPCs;
about 3% to about 15% calcium carbonate;
about 15% to about 50% sodium alginate;
about 0.5% to about 5% total organic acid, comprising one or more of citric, fumaric, malic, glutamic, succinic and tartaric acids;
about 0.1% to about 0.3% sodium lauryl sulfate;
about 0.1% to about 0.3% silicon dioxide;
about 0.5% to about 1% magnesium stearate; and
about 0.5% to about 2% stearic acid.

63. A method for treating Parkinson's disease in a subject, comprising orally administering a composition of claim 1 to the subject in an amount providing a unit dosage of levodopa of about 50 to about 1000 mg at a dosage interval of about 3 to about 24 hours.

64. The method of claim 63, wherein the dosage interval is about 6 to about 24 hours.

65. The method of claim 63, wherein the dosage interval is about 8 to about 12 hours.

66. The method of claim 63, wherein the composition is administered in an amount providing a unit dosage of levodopa of about 100 to about 500 mg.

67. The method of claim 63, wherein trough concentration of levodopa in plasma of the subject does not fall below a minimum threshold concentration of about 300 ng/ml.

68. The method of claim 63, wherein trough concentration of levodopa in plasma of the subject does not fall below a minimum threshold concentration of about 500 ng/ml.

69. A method for treating Parkinson's disease in a subject, comprising (a) identifying a minimum threshold of levodopa concentration in plasma of the subject below which adverse motor effects are observed, and (b) orally administering a composition of claim 16 to the subject in an amount providing a unit dosage of levodopa of about 50 to about 1000 mg at a dosage interval effective to provide a trough concentration of levodopa in plasma of the subject that is not lower than the minimum threshold concentration identified.

70. The method of claim 69, wherein identifying a minimum threshold of levodopa concentration comprises
(i) measuring concentration of levodopa in plasma of the subject during at least one period of levodopa administration;
(ii) recording frequency and/or intensity of adverse motor effects in the subject during the at least one period of levodopa administration;
(iii) correlating the concentration of levodopa in plasma with the frequency and/or intensity of adverse motor effects in the subject; and
(iv) identifying from the resulting correlation a minimum threshold concentration below which adverse motor effects occur at an unacceptable frequency or intensity.

71. A method for reducing motor complications arising from Parkinson's disease therapy with levodopa, the method comprising orally administering the levodopa to the subject in a unit dosage of about 50 to about 1000 mg in the form of a composition of claim 1, at a dosage interval of about 3 to about 24 hours.
